(19) United States
(12) Patent Application Publication
(10) Pub. No.: US 2006/0247462 A1
(43) Pub. Date: Nov. 2, 2006

(54) Production processes of lower aliphatic carboxylic acid alkenyl and alkenyl alcohol

(76) Inventors: Meiko Saihata, Oita (JP); Hiroshi Uchida, Kanagawa (JP)

Correspondence Address: SUGHRUE MION, PLLC, 2100 PENNSYLVANIA AVENUE, N.W., SUITE 800, WASHINGTON, DC, 20037, US

(21) Appl. No.: 10/546,761

(22) PCT Filed: Feb. 25, 2004
(86) PCT No.: PCT/JP04/02214

§ 371 Date: Aug. 25, 2005

Related U.S. Application Data
(60) Provisional application No. 60/453,951, filed on Mar. 13, 2003.

(30) Foreign Application Priority Data
Mar. 7, 2003 (JP) 2003-061494
Apr. 24, 2003 (JP) 2003-119757

(51) Int. Cl. C07C 67/05

(52) U.S. Cl. 560/243

(57) ABSTRACT

A process for producing a lower aliphatic carboxylic acid alkenyl, comprising reacting a lower olefin, a lower aliphatic carboxylic acid and oxygen in a gas phase in the presence of a catalyst comprising a support having supported thereon a catalyst component containing a compound containing alkali metal and/or alkaline earth metal, an element belonging to Group 11 of the Periodic Table or a compound containing at least one of these elements, and palladium, wherein the outflow ratio of the compound containing alkali metal and/or alkaline earth metal is from 1.0 (10−5 to 0.01%/h.

### CROSS-REFERENCE TO RELATED APPLICATION

This application is an application filed under 35 U.S.C. §111(a) claiming benefit pursuant to 35 U.S.C. §119(e)(1) of the filing date of the Provisional Application 60/453,951 filed Mar. 13, 2003, pursuant to 35 U.S.C. §111(b).

### TECHNICAL FIELD

The present invention relates to processes for producing a lower aliphatic carboxylic acid alkenyl and an alkenyl alcohol; and a lower aliphatic carboxylic acid alkenyl and an alkenyl alcohol obtained by the production processes. More specifically, the present invention relates to a process for producing a lower aliphatic carboxylic acid alkenyl from a lower olefin, a lower aliphatic carboxylic acid and oxygen; a lower aliphatic carboxylic acid alkenyl obtained by the production process; a process for producing an alkenyl alcohol by hydrolyzing the above-described lower aliphatic carboxylic acid alkenyl; and an alkenyl alcohol obtained by this production process.

### BACKGROUND ART

In the production process of a lower aliphatic carboxylic acid alkenyl, where a lower aliphatic carboxylic acid alkenyl is obtained by a gas phase reaction starting from a lower olefin, a lower aliphatic carboxylic acid and oxygen, a catalyst comprising a support having supported thereon palladium as the main catalyst component and an alkali metal and/or alkaline earth metal compound as a co-catalyst is widely used. For example, Japanese Unexamined Patent Publication No. 2-91045 (JP-A-2-91045) discloses a process for producing allyl acetate by using a catalyst comprising a support having supported thereon palladium/potassium acetate/copper.

In the production of allyl acetate by using this catalyst system, a phenomenon wherein a compound containing alkali metal and/or alkaline earth metal compound or a component derived from the compound as one component of the catalyst (hereinafter, these are collectively called an “alkali component(s)”) desorbs and flows out from the catalyst during the reaction is seen and this is considered to be one cause bringing about deactivation of the catalyst. The mechanism of desorption is not particularly understood, but one reason for the desorption occurring is believed to be that the lower aliphatic carboxylic acid in the starting material and the alkali component(s) react, producing a new compound (hereinafter referred to as a “lower aliphatic carboxylic acid compound”), and this lower aliphatic carboxylic acid compound is more readily desorbed from the catalyst than the alkali component(s) present in the catalyst.

For the purpose of overcoming this problem, in JP-A-2-91045, at the production of allyl acetate in the presence of a catalyst comprising palladium/potassium acetate/copper, a potassium acetate is added to the supply gas and mixed into the system so as to compensate for the amount of potassium acetate desorbed from the catalyst. Also, in Japanese Unexamined Patent Publication No. 61-238759 (JP-A-61-238759), 20 ppm of potassium acetate is added to the starting material acetic acid at the time of producing allyl acetate in the presence of a palladium/potassium acetate catalyst.

These techniques have a certain effect from the standpoint of preventing the catalyst from reducing in activity due to flowing out of an alkali metal and/or alkaline earth metal compound, particularly potassium acetate at the production of allyl acetate. However, as described in these patent publications, a highly efficient and industrially stable production can sometimes not continue over a long time only by controlling the amount of potassium acetate added to the starting material. More specifically, the potassium acetate added is partially deposited in a reactor, and as a result, a reaction proceeds locally in a part of the catalyst layer, reducing the total reaction yield or the catalyst is partially deteriorated and shortened in the catalyst life. Furthermore, the potassium acetate flowing out from the catalyst is partially deposited, clogging the reaction tube or increasing the flow resistance and it is sometimes difficult to carry out the production stably for a long period of time.

### DISCLOSURE OF INVENTION

An object of the present invention is to provide a process capable of stably producing a lower aliphatic carboxylic acid alkenyl with higher efficiency over a longer period of time.

Another object of the present invention is to provide a process capable of efficiently producing an alkenyl alcohol by hydrolyzing a lower aliphatic carboxylic acid alkenyl produced by the above-described process.

As a result of intensive investigations to attain these objects, the present inventors have found that not only by adding an alkali component(s), which flows out, to the starting material and compensating the component(s), but also by controlling the outflow of an alkali component(s) contained in the catalyst and adding the component(s) in an amount compensating for the outflow, the activity and life of a catalyst can be maintained and a stable operation can be performed over a long time. The present invention has been accomplished based on this finding.

Specifically, the present invention (I) is a process for producing a lower aliphatic carboxylic acid alkenyl, comprising reacting a lower olefin, a lower aliphatic carboxylic acid and oxygen in a gas phase in the presence of a catalyst comprising a support having supported thereon a catalyst component containing (a) a compound containing alkali metal and/or alkaline earth metal, (b) an element belonging to Group 11 of the Periodic Table or a compound containing at least one of these elements, and (c) a palladium, wherein the outflow ratio of (a) the compound containing alkali metal and/or alkaline earth metal, represented by formula (1), is from 1.0×10⁻⁵ to 0.01%/h:
Outflow ratio (%)/h={mass (kg/h) of alkali metal or alkaline earth metal detected/mass (kg) of alkali metal or alkaline earth metal in the entire catalyst packed}×100 (1)

The present invention (II) is a lower aliphatic carboxylic acid alkenyl produced by the production process of the present invention (I).

The present invention (III) is a process for producing an alkenyl alcohol, comprising hydrolyzing the lower aliphatic carboxylic acid alkenyl of the present invention (II) in the presence of an acid catalyst to obtain an alkenyl alcohol, and also includes an alkenyl alcohol produced by this production process.

The present invention having such constitutions comprises, for example, the following matters.

[1] A process for producing a lower aliphatic carboxylic acid alkenyl, comprising reacting a lower olefin, a lower aliphatic carboxylic acid and oxygen in a gas phase in the presence of a catalyst comprising a support having supported thereon a catalyst component containing (a) a compound containing alkali metal and/or alkaline earth metal, (b) an element belonging to Group 11 of the Periodic Table or a compound containing at least one of these elements, and (c) palladium, wherein the outflow ratio of (a) the compound containing alkali metal and/or alkaline earth metal, represented by formula (1), is from 1.0×10⁻⁵ to 0.01%/h:
Outflow ratio (%)/h={mass (kg/h) of alkali metal or alkaline earth metal detected/mass (kg) of alkali metal or alkaline earth metal in the entire catalyst packed}×100 (1)

[2] The production process as described in [1] above, wherein the outflow ratio is from 0.0001 to 0.008%/h.

[3] The production process as described in [1] above, wherein the outflow ratio is from 0.0005 to 0.005%/h.

[4] The production process as described in any one of [1] to [3] above, wherein (a) the compound containing alkali metal and/or alkaline earth metal is a compound containing at least one member selected from the group consisting of lithium, sodium, potassium, cesium, magnesium, calcium and barium.

[5] The production process as described in any one of [1] to [4] above, wherein (a) the compound containing alkali metal and/or alkaline earth metal is a salt of a lower aliphatic carboxylic acid.

[6] The production process as described in [5] above, wherein the salt of a lower aliphatic carboxylic acid is at least one member selected from lithium, sodium, potassium, cesium, magnesium, calcium and barium salts of formic acid, acetic acid, propionic acid, acrylic acid or methacrylic acid.

[7] The production process as described in any one of [1] to [6] above, wherein (b) the element belonging to Group 11 of the Periodic Table or the compound containing at least one of these elements is an element of copper or gold or a compound containing one or more of copper and gold.

[8] The production process as described in any one of [1] to [7] above, wherein a lower olefin, a lower aliphatic carboxylic acid and oxygen are reacted in the presence of water.

[9] A lower aliphatic carboxylic acid alkenyl produced by the production process described in any one of [1] to [8] above.

[10] The production process as described in any one of [1] to [8] above, wherein the lower aliphatic carboxylic acid is acetic acid, the lower olefin is ethylene and the obtained lower aliphatic carboxylic acid alkenyl is vinyl acetate.

[11] Vinyl acetate produced by the production process described in [10] above.

[12] The production process as described in any one of [1] to [8] above, wherein the lower aliphatic carboxylic acid is acetic acid, the lower olefin is propylene and the obtained lower aliphatic carboxylic acid alkenyl is allyl acetate.

[13] Allyl acetate produced by the production process described in [12] above.

[14] A process for producing an alkenyl alcohol, comprising hydrolyzing the lower aliphatic carboxylic acid alkenyl described in [9] above in the presence of an acid catalyst to obtain an alkenyl alcohol.

[15] The production process as described in [14] above, wherein the acid catalyst is an ion exchange resin.

[16] The production process as described in [14] or [15] above, wherein the lower aliphatic carboxylic acid alkenyl is allyl acetate and the obtained alkenyl alcohol is allyl alcohol.

[17] An alkenyl alcohol produced by the production process described in any one of [14] to [16] above.

[18] Allyl alcohol produced by the production process described in [16] above.

### BEST MODE FOR CARRYING OUT THE INVENTION

Preferred embodiments of the present invention are described in detail below.

The compound containing alkali metal and/or alkaline earth metal (a) in the catalyst for use in the present invention (I) is not particularly limited and examples thereof include compounds containing at least one of the elements belonging to Groups 1 and 2 of the Periodic Table according to IUPAC Nomenclature of Inorganic Chemistry, Rules 1989. This compound is preferably a compound containing at least one element selected from the group consisting of lithium, sodium, potassium, cesium, magnesium, calcium and barium, more preferably a salt of a lower aliphatic carboxylic acid, still more preferably at least one member selected from lithium, sodium, potassium, cesium, magnesium, calcium and barium salts of a formic acid, an acetic acid, a propionic acid, an acrylic acid or a methacrylic acid, particularly preferably an acetate, and most preferably a potassium acetate. Also, a salt of an aliphatic carboxylic acid as a starting material of the aliphatic carboxylic acid alkenyl may be used, but the present invention is not limited thereto.

Examples of (b) the element belonging to Group 11 of the Periodic Table according to IUPAC Nomenclature of Inorganic Chemistry, Rules 1989, or the compound containing at least one of these elements in the catalyst for use in the present invention (I) include Group 11 elements and nitrates, carbonates, sulfates, organic acid salts and halides of a Group 11 element(s). This component is preferably one or more element(s) selected from copper and gold or a compound thereof, and most preferably copper alone and/or gold alone.

The palladium (c) in the catalyst for use in the present invention (I) may have any valence number but is preferably metal palladium. The “metal palladium” as used herein means palladium having 0 valence. This palladium can be usually obtained by reducing a divalent and/or tetravalent palladium ion with hydrazine, hydrogen or ethylene as a reducing agent. At this time, the palladium need not be entirely in the metal state. The starting material of (c) the palladium is not particularly limited and metal palladium or a palladium salt capable of converting into metal palladium can be used. Examples of the palladium salt capable of converting into metal palladium include, but are not limited to, palladium chloride, sodium chloropalladate, palladium nitrate and palladium sulfate.

The support used in the catalyst for use in the present invention (I) may be sufficient if it is a commonly employed porous material. Preferred examples thereof include silica, alumina, silica-alumina, kieselguhr, montmorillonite, titania and zirconia, with silica being more preferred. The silica as used herein is not limited to SiO₂, but silica containing impurities may also be used. The shape of the support is not particularly limited and examples thereof include powder, sphere and pellet, although a spherical support is preferred.

The size of the support used is also not particularly limited and the optimal size of the support varies, depending on the shape or reaction style. For example, when the support is spherical, the particle diameter, which is not particularly limited, is preferably from 1 to 10 mm, more preferably from 3 to 8 mm. In the case of performing a reaction by packing the catalyst in a tubular reactor, if the particle diameter is less than 1 mm, a large pressure loss occurs on passing a gas and the gas circulation may not be performed effectively, whereas if the particle diameter exceeds 10 mm, the reaction gas cannot diffuse into the inside of catalyst and the catalytic reaction may not proceed effectively.

As for the pore structure of the support, the average pore diameter is preferably from 0.1 to 1,000 nm, more preferably from 0.2 to 500 nm, still more preferably from 0.5 to 200 nm. If the average pore diameter is less than 0.1 nm, the gas may hardly diffuse, whereas if it exceeds 1,000 nm, the surface area of the support becomes too small and the catalytic activity may decrease.

The ratio between support and (c) palladium is, as the mass ratio, preferably support: (c) palladium=10 to 1,000:1, more preferably support: (c) palladium=30 to 500:1. If the ratio of support and (c) palladium is, in terms of the mass of support, less than support (c) palladium=10:1, the amount of palladium becomes excessively large for the support, resulting in a poor palladium dispersion state and the reaction yield may decrease, whereas if the ratio of support and (c) palladium is, in terms of the mass of support, larger than support: (c) palladium=1,000:1, the mass of support becomes too large and this is not practical.

The ratio among (a) compound containing alkali metal and/or alkaline earth metal, (b) element belonging to Group 11 of the Periodic Table or compound containing at least one of these elements and (c) palladium is, as the mass ratio, preferably (a) compound containing alkali metal and/or alkaline earth metal: (b) element belonging to Group 11 of the Periodic Table or compound containing at least one of these elements: (c) palladium=0.1 to 100:0.001 to 10:1, more preferably (a) compound containing alkali metal and/or alkaline earth metal: (b) element belonging to Group 11 of the Periodic Table or compound containing at least one of these elements: (c) palladium=1 to 50:0.05 to 5:1.

The catalyst for use in the present invention (I) can be obtained by loading (a) a compound containing alkali metal and/or alkaline earth metal, (b) an element belonging to Group 11 of the Periodic Table or a compound containing at least one of these elements and (c) a palladium on a support. In this case, the method for loading the components (a), (b) and (c) is not particularly limited but examples thereof include a method of performing the following steps (1) to (6) in this order:

Step (1):

a step of impregnating a support with an aqueous solution of a salt of palladium and (b) an element belonging to Group 11 of the Periodic Table or a compound containing at least one of these elements to obtain Catalyst Precursor A;

Step (2):

a step of bringing Catalyst Precursor A obtained in the step (1) into contact with an aqueous solution of an alkali metal salt without drying the precursor A to obtain Catalyst Precursor B;

Step (3):

a step of bringing Catalyst Precursor B obtained in the step (2) into contact with a reducing agent such as hydrazine or formalin to obtain Catalyst Precursor C;

Step (4):

a step of water-washing Catalyst Precursor C obtained in the step (3);

Step (5):

a step of bringing Catalyst Precursor C obtained in the step (4) into contact with (a) a compound containing alkali metal and/or alkaline earth metal to obtain a catalyst; and

Step (6):

a step of drying the catalyst obtained in the step (5).

The catalyst for use in the present invention (I) is preferably, for example, a catalyst produced by this method and having a specific surface area of 10 to 250 m²/g and a pore volume of 0.1 to 1.5 ml/g.

The lower olefin for use in the present invention (I) is not particularly limited. The lower olefin is preferably an unsaturated hydrocarbon having from 2 to 4 carbon atoms, more preferably ethylene or propylene. The ethylene and propylene are not particularly limited and in these hydrocarbons, a lower saturated hydrocarbon such as ethane, methane and propane, or a lower unsaturated hydrocarbon such as butadiene, may be mixed. The hydrocarbon is preferably a high-purity unsaturated hydrocarbon.

The lower aliphatic carboxylic acid for use in the present invention (I) is not particularly limited, but is preferably a lower aliphatic carboxylic acid having from 1 to 4 carbon atoms, more preferably formic acid, acetic acid or propionic acid, still more preferably acetic acid. Lower aliphatic carboxylic acids usually available on the market can be used.

The oxygen for use in the present invention (I) is not particularly limited, and may be supplied in the form of being diluted with an inert gas such as nitrogen or carbon dioxide gas, for example, in the form of air, but oxygen having a purity of 99% or more is preferably used.

The ratio among lower aliphatic carboxylic acid, lower olefin and oxygen for use in the present invention (I) is, as the molar ratio, preferably lower aliphatic carboxylic acid:lower olefin:oxygen=1:0.08 to 16:0.01 to 4. In the case where the lower olefin is an ethylene, the ratio is preferably lower aliphatic carboxylic acid:ethylene:oxygen=1:0.2 to 9:0.07 to 2, and in the case where the lower olefin is a propylene, the ratio is preferably lower aliphatic carboxylic acid:propylene:oxygen=1:1 to 12:0.5 to 2.

The reaction starting material gas for use in the present invention (I) contains a lower olefin, a lower aliphatic carboxylic acid and oxygen and, for example, nitrogen, carbon dioxide or rare gas may be used as a diluent, if desired. When the lower olefin, lower aliphatic carboxylic acid and oxygen are denoted as the reaction starting material, the ratio of reaction starting material and diluent is, as the molar ratio, preferably reaction starting material:diluent=1:0.05 to 9, more preferably reaction starting material diluent=1:0.1 to 3.

The reaction starting material gas for use in the present invention (I) is preferably passed through the catalyst at a space velocity of, in the standard state, from 10 to 15,000 hr⁻¹, more preferably from 300 to 8,000 hr⁻¹. If the space velocity is less than 10 hr⁻¹, the heat of reaction may be difficult to remove, whereas if the space velocity exceeds 15,000 hr⁻¹, the equipment required, such as a compressor, becomes too large and this is not practical.

In the reaction starting material gas for use in the present invention (I), from 0.5 to 20 mol % of water can be added. Preferably, from 1 to 18 mol % of water is added. By virtue of the presence of water in the system, although the reasons are not clearly understood, the outflow of (a) the compound containing alkali metal and/or alkaline earth metal from the catalyst decreases. Even if water is added in an amount exceeding 20 mol %, the effect is not enhanced but rather hydrolysis of an alkenyl acetate may proceed. Therefore, it is preferred that a large amount of water not be present.

In the production process of the present invention (I), the reaction of a lower olefin, a lower aliphatic carboxylic acid and oxygen in the presence of a catalyst may be performed in any conventionally known form as long as it is in a gas phase, but the reaction is preferably a fixed-bed flow reaction.

The construction material of the reactor used in performing the production process of the present invention (I) is not particularly limited but a reactor constituted by a material having corrosion resistance is preferred.

In performing the production process of the present invention (I), the reaction temperature is from 100 to 300° C., preferably from 120 to 250° C. If the reaction temperature is less than 100° C., this may disadvantageously cause the reaction to proceed at an excessively low rate, whereas if the reaction temperature exceeds 300° C., the heat of reaction may not be removed and this is not desirable.

In performing the production process of the present invention (I), the reaction pressure is from 0 to 3 MPaG, preferably from 0.1 to 1.5 MPaG. If the reaction pressure is less than 0 MPaG, this may disadvantageously cause reduction in the reaction rate, whereas if the reaction pressure exceeds 3 MPaG, the equipment required, such as reaction tube becomes expensive and this is not practical.

In the present invention (I), the conversion of the lower aliphatic carboxylic acid is preferably 80% or less. The “conversion” as used herein means a value represented by the following formula (2):
Conversion (%)={(amount (mol) of lower aliphatic carboxylic acid at reactor inlet-amount (mol) of lower aliphatic carboxylic acid at reactor outlet)/amount (mol) of lower aliphatic carboxylic acid at reactor inlet}×100 (2)

There is a correlation between the concentration of the lower aliphatic carboxylic acid and the amount of (a) compound containing alkali metal and/or alkaline earth metal deposited in the vicinity of the outlet and as the concentration of the lower aliphatic carboxylic acid decreases, the amount of (a) the compound containing alkali metal and/or alkaline earth metal deposited increases. If the conversion of the lower aliphatic carboxylic acid exceeds 80%, the concentration of the lower aliphatic carboxylic acid decreases in the vicinity of the reactor outlet, causing deposition of (a) the compound containing alkali metal and/or alkaline earth metal and this may block the reaction or cause a reduction in catalytic performance.

In the production process of the present invention (I), the concentration of the lower aliphatic carboxylic acid at the reactor outlet is preferably 0.5 mol % or more. There is a correlation between the concentration of the lower aliphatic carboxylic acid and the amount of (a) the compound containing alkali metal and/or alkaline earth metal deposited in the vicinity of the outlet and as the concentration of the lower aliphatic carboxylic acid decreases, the amount of (a) the compound containing alkali metal and/or alkaline earth metal deposited increases. If the concentration of the lower aliphatic carboxylic acid is less than 0.5 mol %, (a) the compound containing alkali metal and/or alkaline earth metal is caused to deposit and this may result in blockage of reaction or a reduction in catalytic performance.

The outflow ratio specified by formula (1), that is, the ratio of the alkali metal and/or alkaline earth metal in the catalyst for use in the present invention (I) flowing out from the catalyst, is from 1.0×10⁻⁵ to 0.01%/h. If the outflow ratio is less than 1.0×10⁻⁵%/h, the pores of the catalyst may be clogged or the compound containing alkali metal and/or alkaline earth metal may deposit on the catalyst, causing clogging of the reaction tube, and as a result, the production may not be stable. On the other hand, if the outflow ratio exceeds 0.01%/h, the catalytic performance may disadvantageously decrease at a high rate due to the large outflow of the compound containing alkali metal and/or alkaline earth metal. In this case, the activity may be maintained by feeding from the reaction inlet a compound containing alkali metal and/or alkaline earth metal in an amount large enough to compensate for the outflow of the compound containing alkali metal and/or alkaline earth metal, although this is unprofitable.

In formula (1), the mass of alkali metal or alkaline earth metal outflowing is the mass of alkali metal and/or alkaline earth metal contained in the gas at the reactor outlet. The alkali metal element or alkaline earth element as used herein indicates the alkali metal element or alkaline earth metal element contained as a catalyst component in the catalyst.
Outflow ratio (%)/h={mass (kg/h) of alkali metal or alkaline earth metal detected/mass (kg) of alkali metal or alkaline earth metal in the entire catalyst packed}×100 (1)

For example, in the production process of allyl acetate, potassium acetate is generally used as a co-catalyst and the potassium acetate is appropriately added to the reactor even during reaction, because this co-catalyst flows out from the reaction tube during reaction and is contained as potassium or a potassium compound in the gas at the reactor outlet.

The “catalyst packed” as used herein indicates a catalyst packed in the reactor and being in the state before passing a reaction starting material gas. In the case where two or more reactors are present in series or in parallel in one apparatus (process), the total amount of catalyst packed in all reactors is indicated.

The alkali metal element and/or alkaline earth metal element in the gas at the reactor outlet may be detected by any method. Examples thereof include a method of detecting the element as a condensate at the time of separating and purifying the reactor outlet gas and a method of adsorbing the element by contacting the reaction mixture with an ion exchange resin or the like. Specific examples thereof include a method of cooling the reactor outlet gas to an extent of causing condensation, and determining the potassium concentration in the obtained condensate by an analysis method such as induction coupled plasma emission spectroscopic analysis (hereinafter referred to as “ICP spectroscopic analysis”) or atomic absorption method. The determination method using ICP spectroscopic analysis is not particularly limited but, for example, an absolute calibration curve method may be used.

In the outflow ratio used in the present invention (I), the mass of alkali metal or alkaline earth metal in the entire catalyst packed indicates the mass of alkali metal and/or alkaline earth metal in the entire catalyst packed in the reactor, specifically, the mass of alkali metal and/or alkaline earth metal in the packed catalyst before the catalyst is used for the reaction. The mass of alkali metal and/or alkaline earth metal in the catalyst changes during reaction due to outflowing or deposition of the component fed, but the mass of alkali metal and/or alkaline earth metal as used herein is calculated based on the catalyst before reaction.

The outflow ratio (%/h) can be controlled by the reaction conditions such as reaction temperature, reaction pressure and starting material component, and the reaction conditions are set to give an outflow ratio in the desired range. The controlling method is not particularly limited and, for example, the outflow ratio can be increased by elevating the reaction temperature or increasing the ratio of lower aliphatic carboxylic acid in the starting material component.

During the reaction, (a) a compound containing alkali metal and/or alkaline earth metal must be fed from the reactor inlet in an amount large enough to compensate for the mass of the alkali metal and/or alkaline earth metal outflowing. Preferably, the alkali metal and/or alkaline earth metal is added as (a) a compound of alkali metal and/or alkaline earth metal in an amount of 0.01 to 200 mass %, based on the mass of the alkali metal and/or alkaline earth metal outflowing. More preferably, (a) a compound containing alkali metal and/or alkaline earth metal is added in an amount equivalent to or greater than the mass of the alkali metal and/or alkaline earth metal outflowing. Although the reasons are not clearly understood, when (a) a compound containing alkali metal and/or alkaline earth metal is added in an equivalent amount or more, the reaction yield decreases less.

The compound containing alkali metal and/or alkaline earth metal (a) may be added by any method but is preferably added by mixing it in a reaction starting material gas.

The present invention (II) is described below. The present invention (II) is a lower aliphatic carboxylic acid alkenyl produced by the production process of a lower aliphatic carboxylic acid alkenyl of the present invention (I). Since halogen is not added to the reaction system, the lower aliphatic carboxylic acid alkenyl of the present invention (II) is free from mingling of halogen as compared with a lower aliphatic carboxylic acid alkenyl produced by the liquid phase Wacker method and when used as a starting material, problems such as corrosion of equipment less arise due to no mingling of halogen. Furthermore, when this lower aliphatic carboxylic acid alkenyl is used as a starting material, steps such as removal of halogen can be advantageously dispensed with.

The present invention (III) is described below. The present invention (III) is a process for producing an alkenyl alcohol, comprising hydrolyzing the lower aliphatic carboxylic acid alkenyl of the present invention (II) in the presence of an acid catalyst to obtain an alkenyl alcohol, and also includes an alkenyl alcohol produced by this production process.

The lower aliphatic carboxylic acid alkenyl for use in the present invention (III) is not particularly limited as long as it is a lower aliphatic carboxylic acid alkenyl obtained by the process of the present invention (I), and may contain impurities. This lower aliphatic carboxylic acid alkenyl is preferably allyl acetate.

The pressure in the hydrolysis reaction is not particularly limited but the reaction can be performed, for example, at 0.0 to 1.0 MPaG.

The reaction temperature in the hydrolysis reaction is not particularly limited but is preferably from 20 to 300° C., more preferably from 50 to 250° C.

The hydrolysis reaction for use in the present invention (III) can be performed in any reaction system such as gas phase reaction, liquid phase reaction and solid-liquid reaction.

The hydrolysis reaction is preferably performed by adding water to the lower aliphatic carboxylic acid alkenyl so as to elevate the conversion of the lower aliphatic carboxylic acid alkenyl in the hydrolysis reaction. The amount of water added is preferably from 1.0 to 60 mass %, more preferably from 5 to 40 mass %.

Also, the hydrolysis reaction is preferably performed while removing the produced alkenyl alcohol out of the reaction system. The method for removing the alkenyl alcohol out of the reaction system is not particularly limited, but, for example, a method of adding a substance capable of forming an azeotropic mixture with the alkenyl alcohol and removing the alkenyl alcohol while performing distillation during the reaction, may be used.

Examples of the acid catalyst for use in the hydrolysis reaction of the lower aliphatic carboxylic acid alkenyl include organic acids, inorganic acids, solid acids and salts thereof. Specific examples thereof include formic acid, acetic acid, propionic acid, tartaric acid, oxalic acid, butyric acid, terephthalic acid, fumaric acid, heteropolyacid, hydrochloric acid, sulfuric acid, nitric acid, phosphoric acid, hydrobromic acid, hydrofluoric acid, silica alumina, silica titania, silica magnesia, acidic cation exchange resin and sodium salt, potassium salt, magnesium salt and aluminum salt thereof. Among these, solid acidic cation exchange resin is most preferred in view of easy separation from the alkenyl alcohol after reaction or acidity. This resin is more preferably, for example, an ion exchange resin where an acidic active functional group such as sulfonic acid group is bonded to the styrene•divinylbenzene resin skeleton.

The apparatus of performing the hydrolysis reaction for use in the present invention (III) is not particularly limited, but a fixed bed flow-type reactor is preferred. When two or more reactor units are used in parallel, a constant amount of an alkenyl alcohol can be continuously obtained and therefore, this is preferred.

In the present invention (III), the method of producing an alcohol in a fixed bed flow-type reactor by using an acidic cation exchange resin as the hydrolysis catalyst is not particularly limited, but a method of causing an ascending flow to convey a reaction solution containing the lower aliphatic carboxylic acid alkenyl and water from the bottom of the reactor into the reactor system is preferred. In this case, the coagulation of ion exchange resin and the drifting of reaction starting material, which may occur in the case of passing the reaction solution from the top to the bottom, can be suppressed.

The alkenyl alcohol of the present invention (III) is described below. This alcohol is advantageously free from mingling of halogen, because the lower aliphatic carboxylic acid alkenyl as the starting material does not contain halogen. Therefore, when this alcohol is used as a starting material, steps such as removal of halogen giving rise to corrosion of equipment can be dispensed with and the process can be advantageously simplified.

The present invention is described in greater detail below by referring to Examples. However, the present invention is not limited thereto.

The reactor outlet gas was analyzed by the following methods.

1. Ethylene

An absolute calibration curve method was employed for analysis, where 50 ml of the outflow gas was sampled and the entire amount of the gas flowed into a 1-ml gas sampler attached to a gas chromatograph and was analyzed under the following conditions.
Gas chromatography:
   gas chromatograph (GC-14B manufactured by Shimadzu Corporation) with a gas sampler (MGS-4, measuring tube: 1 ml) for Shimadzu Gas Chromatograph
Column: packed column Unibeads IS, length: 3 m
Carrier gas: helium (flow rate: 20 ml/min)
Temperature conditions:
   The temperature of detector and vaporization chamber was 120° C. and the column temperature was constantly 65° C.
Detector: TCD (He pressure: 70 kPaG, current: 90 mA)
   2. Propylene

An absolute calibration curve method was employed for analysis, where 50 ml of the outflow gas was sampled and the entire amount of the gas flowed into a 1-ml gas sampler attached to a gas chromatograph and was analyzed under the following conditions.
Gas chromatography:
   gas chromatograph (GC-7B manufactured by Shimadzu Corporation) with a gas sampler (MGS-4, measuring tube: 1 ml) for Shimadzu Gas Chromatograph
Column: packed column Unibeads IS, length: 3 m
Carrier gas: helium (flow rate: 35 ml/min)
Temperature conditions:
   The detector temperature was 100° C., the vaporization chamber temperature was 140° C. and the column temperature was constantly 140° C.
Detector: TCD (He pressure: 125 kPaG, current: 125 mA)
   3. Oxygen

An absolute calibration curve method was employed for analysis, where 50 ml of the outflow gas was sampled and the entire amount of the gas was flowed into a 1-ml gas sampler attached to a gas chromatograph and analyzed under the following conditions.
Gas chromatography:
   gas chromatograph (GC-14B manufactured by Shimadzu Corporation) with a gas sampler (MGS-4, measuring tube: 1 ml) for Shimadzu Gas Chromatograph
Column: MS-5A IS, 60/80 mesh (3 mmφ×3 m)
Carrier gas: helium (flow rate: 20 ml/min)
Temperature conditions:
   The temperature of detector and vaporization chamber was 110° C. and the column temperature was constantly 70° C.
Detector: TCD (He pressure: 70 kPaG, current: 100 mA)
   4. Acetic Acid

An internal standard method was employed for analysis, where 1 ml of 1,4-dioxane was added as the internal standard to 10 ml of the reaction solution and 0.2 μl of the resulting analysis solution was injected and analyzed under the following conditions.
Gas chromatography:
   GC-14B manufactured by Shimadzu Corporation
Column: packed column Thermon 3000 (length: 3 m, inner diameter: 0.3 mm)
Carrier gas: nitrogen (flow rate: 20 ml/min)
Temperature conditions:
   The temperature of detector and vaporization chamber was 180° C. and the column temperature was kept at 50° C. for 6 minutes from the start of analysis, then elevated to 150° C. at a temperature rising rate of 10° C./min and kept at 150° C. for 10 minutes.
Detector: FID (H₂ pressure: 40 kPaG, air pressure: 100 kPaG)
   5. Vinyl Acetate

An internal standard method was employed for analysis, where 1 g of n-propyl acetate was added as the internal standard to 6 g of the reaction solution and 0.3 μl of the resulting analysis solution was injected and analyzed under the following conditions.
Gas chromatography:
   GC-9A manufactured by Shimadzu Corporation
Column: capillary column TC-WAX (length: 30 m, inner diameter: 0.25 mm, film thickness: 0.5 μm)
Carrier gas: nitrogen (flow rate: 30 ml/min)
Temperature conditions:
   The temperature of detector and vaporization chamber was 200° C. and the column temperature was kept at 45° C. for 2 minutes from the start of analysis, then elevated to 130° C. at a temperature rising rate of 4° C./min, kept at 130° C. for 15 minutes, again elevated to 200° C. at a temperature rising rate of 25° C./min and kept at 200° C. for 10 minutes.
Detector: FID (H₂ pressure: 60 kPaG, air pressure: 100 kPaG)
   6. Allyl Acetate

An internal standard method was employed for analysis, where 1 g of pentyl acetate was added as the internal standard to 25 g of the reaction solution and 0.3 μl of the resulting analysis solution was injected and analyzed under the following conditions.
Gas chromatography:
   GC-9A manufactured by Shimadzu Corporation
Column: capillary column TC-WAX (length: 30 m, inner diameter: 0.25 mm, film thickness: 0.5 μm)
Carrier gas: nitrogen (flow rate: 30 ml/min)
Temperature conditions:
   The temperature of detector and vaporization chamber was 200° C. and the column temperature was kept at 45° C. for 2 minutes from the start of analysis, then elevated to 130° C. at a temperature rising rate of 4° C./min, kept at 130° C. for 15 minutes, again elevated to 200° C. at a temperature rising rate of 25° C./min and kept at 200° C. for 10 minutes.
Detector: FID (H₂ pressure: 60 kPaG, air pressure: 100 kPaG)
   7. Allyl Alcohol

An internal standard method was employed for analysis, where 200 μl of n-amine acetate was added as the internal standard to 10 ml of the reaction solution, and 0.1 μl of the resulting analysis solution was injected and analyzed under the following conditions.
Gas chromatography:
   GC-14B manufactured by Shimadzu Corporation
Column: packed column Thermon 3000 (length: 3 m, inner diameter: 0.3 mm)
Carrier gas: nitrogen (flow rate: 2.0 ml/min)
Temperature conditions:
   The temperature of detector and vaporization chamber was 180° C. and the column temperature was kept at 45° C. for 5 minutes from the start of analysis, then elevated to 130° C. at a temperature rising rate of 7° C./min and kept at 130° C. for 13 minutes.
Detector: FID (H₂ pressure: 98 kPaG, air pressure: 98 kPaG)

### EXAMPLE 1

Preparation of Catalyst A:

Sodium chloropalladate crystal (56.4 mmol), 8.50 mmol of cupric chloride dihydrate and 18.4 mmol of zinc chloride were dissolved in pure water and the resulting solution was measured to 97% of the water absorption amount of the support.

The aqueous metal salt solution obtained above was uniformly impregnated into a silica support (KA-160 produced by Sud-chemi AG) which was previously dried at 110° C. for 4 hours.

Subsequently, sodium metasilicate nonahydrate was dissolved in pure water and the amount of the solution was adjusted to 2 times the water absorption amount of the support. The resulting solution was added to the impregnated support and left standing at room temperature for 20 hours to obtain a catalyst.

To this solution, 720 mmol of hydrazine monohydrate was further added and after stirring at room temperature for 4 hours, the catalyst was washed with pure water and dried by a hot air dryer at 110° C. for 4 hours.

Thereafter, 509 mmol of potassium acetate was dissolved in pure water and the resulting solution was measured to about 97% of the water absorption amount of the catalyst. This solution was uniformly loaded on the catalyst and then dried at 110° C. for 4 hours to obtain Catalyst A for reaction.

Reaction:

The obtained catalyst (20 ml) was packed in a stainless steel-made reaction tube having an inner diameter of 21.4 mm and by supplying a mixed gas containing 30 mol % of propylene, 7.0 mol % of oxygen, 5.5 mol % of acetic acid, 14.0 mol % of water and 43.5 mol % of nitrogen, the reaction was performed at a reaction temperature of 135° C. and a pressure of 0.8 MPaG. The results are shown in Table 2. The concentration of each component in the reaction solution was measured by using a gas chromatography analyzer. The outflow ratio (%/h) was calculated according to formula (1) from the weight of potassium in the condensate obtained by cooling the gas at the outlet of the reaction tube and the weight of potassium in the catalyst before use.

Analysis of Potassium:

1. Analysis of Catalyst Before Use

The catalyst before use was finely ground in an agate mortar and then dried at 110° C. for 2 hours to prepare a powder sample. To 1 g of the powder sample, 100 ml of pure water was added and 10 ml of 35% hydrochloric acid was further added. Thereafter, the sample was boiled in a sand bath for 2 hours and then allowed to cool and thereto, pure water was added to a quantity of 500 ml. After filtering, the filtrate was subjected to ICP spectroscopic analysis under the following conditions by using SPS1700HUR manufactured by Seiko Instruments Inc. and the amount of potassium was calculated.
Measuring method: absolute calibration curve method
Photometric height: 15 nm
High-frequency output: 1.3 kw
Carrier pressure: 0.22 MPa
Plasma flow rate: 16 L/min
Photomultiplier voltage: H
Auxiliary flow rate: 0.5 L/min

2. Analysis of Potassium Detected After Reactor Outlet

The condensate obtained by recovering the reaction gas at normal temperature and atmospheric pressure was subjected to ICP spectroscopic analysis under the same conditions as in “1. Analysis of Catalyst Before Use” above, and the amount of potassium was calculated.

The results are shown in Table 2.

### EXAMPLE 2

Preparation of Catalyst B:

An aqueous solution containing 47.0 mmol of sodium chloropalladate and 10.2 mol of chloroauric acid tetrahydrate was dissolved in pure water and the resulting solution was measured to 90% of the water absorption amount of the support.

The aqueous metal salt solution obtained above was uniformly impregnated into a silica support (KA-160) which was previously dried at 110° C. for 4 hours.

Subsequently, 135 mmol of sodium metasilicate nonahydrate was dissolved in pure water and the amount of the solution was adjusted to 2 times the water absorption amount of the support. The resulting solution was added to the impregnated support and left standing at room temperature for 20 hours to obtain a catalyst.

To this solution, 538 mmol of hydrazine monohydrate was further added and after stirring at room temperature for 4 hours, the catalyst was washed with pure water and dried by a hot air dryer at 110° C. for 4 hours.

Thereafter, 33 g of potassium acetate was dissolved in pure water and the resulting solution was measured to about 90% of the water absorption amount of the catalyst. This solution was uniformly loaded on the catalyst and then dried at 110° C. for 4 hours to obtain Catalyst B for reaction.

Then, the reaction was performed in the same manner as in Example 1, except that the reaction conditions were changed as shown in Table 1. Also, the analysis was performed in the same manner as in Example 1. The results are shown in Table 2.

### EXAMPLE 3

The reaction was performed in the same manner as in Example 1 except for changing the reaction conditions as shown in Table 1 to give an outflow ratio of 1.2×10⁻⁴%/h. The results are shown in Table 2.

### EXAMPLE 4

The allyl acetate reaction solution obtained in Example 1 was hydrolyzed at 80° C. and 0.5 MPaG by using an ion exchange resin (Diaion SK104H, produced by Mitsubishi Chemical Corporation) to obtain an allyl alcohol. The conversion was 98% and the selectivity was 98%.

### COMPARATIVE EXAMPLE 1

The reaction was performed in the same manner as in Example 1 except for changing the reaction conditions to give an outflow ratio of 5×10⁻⁶%/h. The results are shown in Table 2.

After the reaction, the catalyst was drawn out, and as a result, potassium acetate was found to be present in the vicinity of the reaction tube outlet.

TABLE 1

| | | | Reaction | Reaction | Reaction Gas | | | | | |
|---|---|---|---|---|---|---|---|---|---|---|
| | | SV | Temperature | Pressure | Olefin | | Oxygen | Acetic Acid | Water | Nitrogen |
| | Catalyst | h⁻¹ | ° C. | MPaG | mol % | | | | | |
| Example 1 | A | 1600 | 135 | 0.8 | propylene | 30 | 7 | 5.5 | 14 | 43.5 |
| Example 2 | B | 2700 | 150 | 0.8 | ethylene | 60 | 6.5 | 17 | 1.3 | 15.2 |
| Example 3 | A | 1600 | 145 | 0.8 | propylene | 30 | 6.5 | 5 | 14 | 44.5 |
| Comparative | A | 1600 | 145 | 0.8 | propylene | 30 | 7 | 5 | 14 | 45 |
| Example 1 | | | | | | | | | | |

TABLE 2

| | Alkenyl | | Alkenyl | | | |
|---|---|---|---|---|---|---|
| | Acetate, | | Acetate, | | Conversion | |
| | STY | | Sel | | of Acetic | |
| | after | after | after | after | Acid | Outflow |
| | 5 h | 2400 h | 5 h | 2400 h | after 5 h | Ratio |
| | g/L-cat | | % | | % | %/h |
| Example 1 | 355.7 | 320.1 | 92 | 91 | 70.3 | 6.5 × 10⁻⁴ |
| Example 2 | 349.7 | 301.4 | 92 | 90 | 26 | 2.2 × 10⁻⁴ |
| Example 3 | 366.6 | 311.6 | 94 | 90 | 78 | 1.2 × 10⁻⁴ |
| Comparative | 380.7 | 311.9 | 94 | 83 | 81 | 5 × 10⁻⁶ |
| Example 1 | | | | | | |

### INDUSTRIAL APPLICABILITY

According to the present invention, in producing a lower aliphatic carboxylic acid alkenyl from a lower aliphatic carboxylic acid, a lower olefin and oxygen, the compound containing alkali metal and/or alkaline earth metal as a catalyst component is controlled in the outflowing during reaction, so that production can proceed stably for a long time without impairing the catalytic activity.

1. A process for producing a lower aliphatic carboxylic acid alkenyl, comprising reacting a lower olefin, a lower aliphatic carboxylic acid and oxygen in a gas phase in the presence of a catalyst comprising a support having supported thereon a catalyst component containing (a) a compound containing alkali metal and/or alkaline earth metal, (b) an element belonging to Group 11 of the Periodic Table or a compound containing at least one of these elements, and (c) palladium, wherein the outflow ratio of (a) the compound containing alkali metal and/or alkaline earth metal, represented by formula (1), is from 1.0×10⁻⁵ to 0.01%/h:
Outflow ratio (%)/h={mass (kg/h) of alkali metal or alkaline earth metal detected/mass (kg) of alkali metal or alkaline earth metal in the entire catalyst packed}×100 (1)

2. The production process as claimed in claim 1, wherein the outflow ratio is from 0.0001 to 0.008%/h.

3. The production process as claimed in claim 1, wherein the outflow ratio is from 0.0005 to 0.005%/h.

4. The production process as claimed in any one of claims 1 to 3, wherein (a) the compound containing alkali metal and/or alkaline earth metal is a compound containing at least one member selected from the group consisting of lithium, sodium, potassium, cesium, magnesium, calcium and barium.

5. The production process as claimed in any one of claims 1 to 4, wherein (a) the compound containing alkali metal and/or alkaline earth metal is a salt of a lower aliphatic carboxylic acid.

6. The production process as claimed in claim 5, wherein the salt of a lower aliphatic carboxylic acid is at least one member selected from lithium, sodium, potassium, cesium, magnesium, calcium and barium salts of formic acid, acetic acid, propionic acid, acrylic acid or methacrylic acid.

7. The production process as claimed in any one of claims 1 to 6, wherein (b) the element belonging to Group 11 of the Periodic Table or the compound containing at least one of these elements is an element of copper or gold or a compound containing one or more of copper and gold.

8. The production process as claimed in any one of claims 1 to 7, wherein a lower olefin, a lower aliphatic carboxylic acid and oxygen are reacted in the presence of water.

9. A lower aliphatic carboxylic acid alkenyl produced by the production process as set forth in any one of claims 1 to 8.

10. The production process as claimed in any one of claims 1 to 8, wherein the lower aliphatic carboxylic acid is acetic acid, the lower olefin is ethylene and the obtained lower aliphatic carboxylic acid alkenyl is vinyl acetate.

11. Vinyl acetate produced by the production process as set forth in claim 10.

12. The production process as claimed in any one of claims 1 to 8, wherein the lower aliphatic carboxylic acid is acetic acid, the lower olefin is propylene and the obtained lower aliphatic carboxylic acid alkenyl is allyl acetate.

13. Allyl acetate produced by the production process as set forth in claim 12.

14. A process for producing an alkenyl alcohol, comprising hydrolyzing the lower aliphatic carboxylic acid alkenyl as set forth in claim 9 in the presence of an acid catalyst to obtain an alkenyl alcohol.

15. The production process as claimed in claim 14, wherein the acid catalyst is an ion exchange resin.

16. The production process as claimed in claim 14 or 15, wherein the lower aliphatic carboxylic acid alkenyl is allyl acetate and the obtained alkenyl alcohol is allyl alcohol.

17. Alkenyl alcohol produced by the production process as set forth in any one of claims 14 to 16.

18. Allyl alcohol produced by the production process as set forth in claim 16.
